(19) United States
(12) Patent Application Publication
(10) Pub. No.: US 2001/0021372 A1
(43) Pub. Date: Sep. 13, 2001

(54) Apparatus having partially gold-plated surface

(76) Inventors: Tore Omtveit, Eiksmarka (NO); Ann-Kristin Honerud, Oslo (NO)

Correspondence Address: AMERSHAM PHARMACIA BIOTECH INC., PATENT DEPARTMENT, P O BOX 1327, 800 CENTENNIAL AVENUE, PISCATAWAY, NJ, 088551327

(21) Appl. No.: 09/775,959

(22) Filed: Feb. 2, 2001

Related U.S. Application Data
(63) Continuation of application No. PCT/GB99/02741, filed on Aug. 18, 1999.
(60) Provisional application No. 60/101,224, filed on Sep. 21, 1998.

(30) Foreign Application Priority Data
Aug. 18, 1998 (GB) 9818021.9

(51) Int. Cl. A61K 9/00

(52) U.S. Cl. 424/43

(57) ABSTRACT

A change in the surface properties of apparatus for pharmaceutical preparation results from steam sterilization. This change may be avoided by the use of apparatus with more hydrophilic post-sterilization surfaces, e.g. those of gold.

Hence there is provided a process for the preparation of a mixture comprising a discontinuous phase in a continuous phase which comprises using a mixing apparatus having a non-porous mixing surface which is repellant to the discontinuous phase and/or attractive to the continuous phase when the continuous phase is liquid or which is attractive to the discontinuous phase when the continuous phase is gaseous, whereby to improve the efficacy of the preparation of said mixture and/or to reduce physical degradation of said mixing surface, said surface if hydrophilic being more hydrophilic after sterilization than is stainless steel and if hydrophobic being more hydrophobic after sterilization than is stainless steel.

This is a continuation of International Application PCT/GB99/02741, with an international filing date of Aug. 18, 1999.

### BACKGROUND OF INVENTION

This invention relates to improvements in and relating to apparatus used for production of a pharmaceutical product comprising a continuous aqueous phase containing a discontinuous water-immiscible phase, in particular high shear mixer apparatus.

In the preparation of pharmaceuticals, it is important that the apparatus used should be capable of sterilization. This is generally achieved for large scale apparatus by steam sterilization, i.e. by passing steam, usually superheated steam, through the apparatus between production runs.

Much of the large scale apparatus used in pharmaceutical production is made of stainless steel because of its chemical inertness, corrosion resistance, ease of production and simplicity of sterilization.

We have now found however that mixing apparatus made of stainless steel and subjected in use to high shear forces may suffer a reduction in efficacy following steam sterilization. This is particularly critical in the case of rotor:stator mixers used to generate lipid-membraned vesicles for use as pharmaceuticals, e.g. gas containing vesicles for use as ultrasound contrast agents, where the steam sterilization results in a reduction in vesicle yield.

### SUMMARY OF INVENTION

We have found that a change in surface properties of the apparatus surfaces results from steam sterilization and that this may be reduced or avoided by the use of apparatus with more hydrophilic post-sterilisation surfaces, e.g. gold surfaces.

Thus, one aspect the invention provides a process for the preparation of a mixture comprising a discontinuous phase in a continuous phase which comprises using a mixing apparatus having a non-porous mixing surface which is repellant to the discontinuous phase and/or attractive to the continuous phase when the continuous phase is liquid or which is attractive to the discontinuous phase when the continuous phase is gaseous, whereby to improve the efficacy of the preparation of said mixture and/or to reduce physical degradation of said mixing surface, said surface if hydrophilic being more hydrophilic after sterilisation than is stainless steel and if hydrophobic being more hydrophobic after sterilisation than is stainless steel.

Additionally, the invention provides a process for the preparation of a product, preferably a pharmaceutical product, comprising a continuous aqueous phase containing a discontinuous water-immiscible phase, said process comprising mixing an aqueous material and a water-immiscible material in a mixing apparatus, characterized in that at least part of a non-porous surface of said apparatus contacting said aqueous and water-immiscible materials is formed of a substance which when sterilized (e.g. steam sterilized) is more hydrophilic than sterilized steel, e.g. of gold or of a substance as hydrophilic as or more hydrophilic than gold.

For the purposes of comparison of surface hydrophilicity, it is preferred to determine receding water contact angle at 25 EC after a steam sterilization cycle for 1 hour. More preferably it is determined after repeated use (ie. homogenization) and sterilization cycles, e.g. after at least two such cycles, preferably after 3 to 10 cycles.

The process of the invention may yield a ready-for-use pharmaceutical composition directly as a result of the operation of the mixing apparatus or alternatively further steps may be required in the process. Such steps may include dilution, concentration, sterilization, lyophilization, addition of further active or non-active ingredients (e.g. diluents, antioxidants, flavours, colours, stabilizers, suspending agents, etc.), dispersion and size separation, for example as occurs in mixing apparatuses such as extruders as described in more detail below. Likewise, the pharmaceutical product produced by the process of the invention may be an intermediate in the production of or a component for a pharmaceutical composition.

Preferably such compositions are vesicle containing diagnostic contrast media (e.g. X-ray, MR or most preferably ultrasound contrast media) or vesicle containing therapeutic compositions (e.g. compositions where a therapeutic agent, e.g. a cytotoxic agent, is contained within vesicles such as liposomes or micelles).

Since the mixing apparatus is used for production of a pharmaceutical product, the surfaces contacting the materials being mixed are preferably sterile and the apparatus is preferably sealed. The part of the surface of the mixing apparatus which is of the more hydrophilic substance preferably includes at least part of the mixing surfaces, i.e. the portions of the apparatus surface responsible for the mixing action.

Such mixing surfaces may be in motion or may be static during the operation of the mixing apparatus, e.g. they may be on mixing paddles or blades, on rotors in rotor:stator devices, etc. or they may be on atomisers (e.g. spray nozzles), expansion nozzles, static mixers, deflector/diffuser plates, sonicator heads or other sonicator surfaces, or on stators in rotor:stator devices.

In one embodiment, the mixing surface may form part of a mixing apparatus such as an extruder as described in more detail below. Such extruders are especially suitable for the production of emulsions and work advantageously when some of the surfaces of the extruder which contact the reaction mixture are coated with gold. Such extruders may also reduce problems associated with filter clogging. Desirably however such surfaces are ones over which the materials being mixed flow at a rate which is higher than that at which they flow over other surfaces in the mixing apparatus.

### BRIEF DESCRIPTION OF THE FIGURES

FIG. 1 is a cross-sectional drawing of a rotor:stator mixer.

FIG. 2 is a perspective view of the rotor and stator stage in the apparatus of FIG. 1.

FIG. 3 is a cross-sectional drawing of an extruder suitable for use in the preparation of liposome suspensions.

FIG. 4 presents a representation of a cross sectional drawing of an extruder suitable for use as a mixing apparatus according to the invention.

### DETAILED DESCRIPTION OF THE INVENTION

The mixing apparatus will generally comprise one or more chambers (mixing chambers) in which mixing occurs. In the process of the invention a more hydrophilic surface is preferably present in one or more, preferably all, of such chambers.

If desired, all surfaces in the mixing apparatus in contact with the materials being mixed (i.e. all contact surfaces) may be of the hydrophilic substance. Generally however only mixing surfaces (rather than chamber surfaces and inlet and outlet duct surfaces) will be of the hydrophilic substance.

The hydrophilic substance may be any material capable of meeting the mechanical and chemical demands of the portion of the mixing apparatus in which it occurs and which after steam sterilization is more hydrophilic than steel (e.g. stainless steel) after, for example, equivalent steam sterilization. Hydrophilicity in this regard may be compared by comparing the contact angle between pure water and the hydrophilic substance or steel surfaces. The larger the contact angle the less hydrophilic is the surface.

In general the hydrophilic substance will preferably have a receding contact angle (measured at 25 EC after sterilization for 1 hour with steam) of less than 55 E, preferably less than 45 E, more preferably less than 30 E. One example of a suitable hydrophilic material is gold and the water receding contact angle for gold steam sterilized for 1 hour is about 26 E. By contrast, the receding contact angle for stainless steel steam sterilized for 1 hour is 60 E. Contact angles may be measured as described by Hansen et al. in J. Colloid Interface Sci. 141 (1991) and by Hansen in J. Colloid Interface Sci. 160: 209 (1993), both of which are incorporated herein by reference.

The hydrophilic substance may be used to form entire components of the mixing apparatus; more generally however the hydrophilic substance will form a surface on a substrate, e.g. of steel or other metal or of a ceramic. The hydrophilic substance may be a material deposited on the substrate (e.g. by electroplating, vapour deposition, fusion, lamination, film deposition, painting, etc.) or alternatively it may be a material produced by transformation of a substrate surface, e.g. by plasma treatment or particle bombardment. While gold is a particularly preferred hydrophilic substance, alternative substances include ceramics, enamels, glass and vitreous glazes. The hydrophilic material may thus comprise a surface layer of a thickness sufficient to survive normal operation of the mixing apparatus. Suitable thicknesses will depend on the nature of the hydrophilic material and geometry and nature of mixer and mixing surface. However, suitable thicknesses generally will be in the range 0.5 to 50 μm, preferably 2 to 30 μm, e.g. 3 to 20 μm, most preferably about 3 μm.

The hydrophilic surfaces, and indeed all other contact surfaces in the mixing apparatus used according to the invention will preferably be smooth and also preferably non water-porous. Smoothness in this regard is as measurable by touch as hydrophilic surfaces may be created, even on steel substrates, by particle bombardment, e.g. by glass blasting, to create roughness at a nanometer scale (nano-roughness). In general, any roughness in surfaces should be at a scale smaller than the droplet size produced by the process.

The mixing apparatus used according to the invention may be any apparatus capable of generating a composition comprising a continuous aqueous phase containing a discontinuous non-water miscible phase, e.g. an emulsion, dispersion, foam, suspension, etc. Such apparatus will generally be provided with a power source capable of causing the mixing effect, e.g. a pressure source or pump or a motor capable of moving mixing surfaces in the apparatus. The apparatus will not generally be one in which the mixing effect is achieved by the effort of the operator, e.g. by manual stirring. Moreover the apparatus will preferably be arranged for operation under computer control or under on/off control by an operator.

The use of gold surfaces in pharmaceutical mixing apparatus is novel and such apparatus having gold contact surfaces forms a further aspect of the invention. Viewed from this aspect the invention provides a pharmaceutical mixing apparatus, preferably adapted for steam sterilization (e.g. by provision of steam inlet and drainage ports), having a surface which in use contacts the material being mixed therein, wherein at least part of said surface is of gold, e.g. is gold plated.

The mixing apparatus of or used according to the invention may be any of the conventional types of mixing apparatus, e.g. homogenisers, atomisers, extruders, sonicators, static mixers, expansion nozzles, capmixers, shakers, paddle or blade mixers, etc. However, the apparatus is preferably one in which the material being mixed is subjected to high shear forces, e.g. high shear homogenisers and sonicators and in particular rotor:stator mixers (e.g. as supplied by Ytron or as described in WO99/08782).

In rotor:stator mixers, the rotor and stator surfaces over which the material being mixed passes are preferably of a hydrophilic material or provided with a surface of a hydrophilic material. Likewise in atomisers (e.g. in spray mixers) the lining of the spray nozzle is preferably of a hydrophilic material or provided with a hydrophilic material surface although it is envisaged that the lining of the spray nozzle may also be a hydrophobic material or provided with a hydrophobic material.

The mixing apparatus of or used according to the invention may likewise be provided with inlet and outlet ports for the materials to be mixed and the resulting mixture, etc. and optionally with inlet and outlet ports for flushing or cleaning or sterilizing materials. Otherwise the apparatus is preferably sealed or sealable.

invention are preferably fluids whereby an The materials being mixed using the process of the emulsion, suspension or dispersion is produced on mixing. The compositions produced on mixing (and any subsequent processing steps) are preferably diagnostic imaging contrast media (particularly vesicular ultrasound, X-ray or MR contrast media, for example dispersions of gas microbubbles or of MR or X-ray contrast agent containing vesicles) or therapeutic compositions (e.g. dispersions of therapeutic agent—containing vesicles). Particularly preferably, the materials being mixed comprise an aqueous liquid, a vesicle (e.g. liposome or micelle or gas microbubble) membrane forming agent (e.g. a lipid, in particular a phospholipid or a mixture of phospholipids) and optionally and preferably a gas or gas-precursor (e.g. air, nitrogen, sulphur hexafluoride or a fluorocarbon, for example a C₃₋₆ perfluorocarbon). By gas-precursor is meant a material or mixture of materials which is at least partially in the gaseous phase at 37° C. and atmospheric pressure or which is reactive to generate a gas in vivo. Examples of suitable lipids, gases and gas precursors are described in WO 97/29783 (Nycomed).

It is to be understood that the basic processes and apparatus of the invention may however be extended to the mixing of non-pharmaceutical compositions, e.g. cosmetics and foodstuffs, and even to the production of droplet-in-gas dispersions of aqueous or hydrophilic liquids, e.g. as in spray-dryers and prilling towers. In the production of droplet-in-gas dispersions (sprays), the hydrophilic surface will preferably be provided on the internal surface of the spray nozzle. Such uses and apparatus therefor form further aspects of the invention.

It is also to be understood that the basic processes and apparatus of the invention may likewise be applied to the production of products comprising a water-immiscible continuous phase containing an at least partially water-miscible discontinuous phase (e.g. water-in-oil emulsions) as well as to the production of droplet-in-gas dispersions (sprays) of water-immiscible liquids (e.g. of hydrocarbon fuels for example as sprayed in injection nozzles in diesel engines). In such cases, it is preferred to use in place of a hydrophilic surface, a hydrophobic surface, i.e. one which following steam sterilization is more hydrophobic than steam sterilized steel (e.g. with a water contact angle above 90°), for example a hydrophobic polymer such as a polyolefin or a fluorinated polyolefin such as PTFE, or a substance as hydrophobic as PTFE or more hydrophobic than PTFE. However, it is envisaged that a hydrophilic surface may also be effective. Such uses and apparatus therefor form still further aspects of the present invention.

### EXAMPLES

Certain preferred embodiments of the invention will now be described further. They are presented by way of example only, and are not intended to be illustrative of all embodiments.

### Example 1

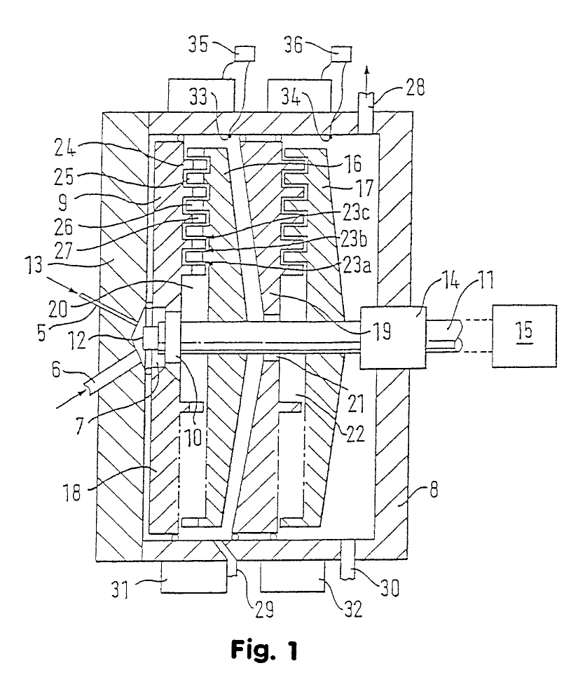

Referring to FIG. 1, there is shown a rotor: stator mixer. Gas (e.g. a perfluorocarbon such as perfluorobutane) and liquid (e.g. an aqueous phospholipid mixture) are introduced through inlets 5 and 6 respectively into premixing chamber 7, the walls of which are defined by a concave section of housing 8, first stator element 9, and the tip 10 of rotor drive shaft 11.

Tip 10 of rotor drive shaft 11 carries a flange 12 (seen side-on) which serves to mix gas and liquid in pre-mix chamber 7.

Housing 8 provides a cylindrical chamber and has a cup-shaped portion 8 and an end cap 13 with rotor drive shaft 11 entering through the base of portion 8 and sealed by a double mechanical seal 14. Drive shaft 11 is rotated for example at speeds of up to 8000 rpm by externally positioned motor 15 and rotates first rotor 16 and second rotor 17 which are in interlocking engagement with first stator 18 and second stator 19. The rotors and stators are gold plated to a thickness of 2 to 30 μm and have an external diameter of about 110 mm.

Premix chamber 7 communicates with first mixing chamber 20 which is defined by surfaces of housing 8, first stator 18, and second stator 19 and which has an outlet 21 which corresponds to the inlet to second mixing chamber 22. Second mixing chamber 22 is defined by surfaces of second stator 19 and housing 8.

In first mixing chamber 20, material from premixing chamber 7 passes radially outward through shear force zones 23a,b,c etc. between cylindrical extensions 24,25 of first stator 18 and first rotor 16 through fluid passageways defined by axial slots 26,27 in first rotor and first stator. Conveniently each rotor:stator assembly defines about 12 to 14 such shear force zones and the radial clearance between the cylindrical extensions is conveniently about 250 μm. At the periphery of the mixing chamber the material being mixed then passes radially inward to pass from first mixing chamber to second mixing chamber through outlet 21.

The cylindrical extensions of rotor and stator are not shown in the lower portion of FIG. 1 and the slots in cylindrical extensions of second rotor and second stator are likewise not shown.

In second mixing chamber 22, the mixture passes radially outward between second rotor and second stator before leaving through outlet 28 as a gas microbubble suspension.

First and second mixing chambers 20 and 22 are provided with drainage ports 29 and 30 along their lower boundary. These drainage ports may be connected to a steam trap and drain, e.g. as is conventional in pharmaceutical manufacturing apparatus. For sterilization, steam (generally superheated steam) is introduced into the mixing chambers through inlet 5. Liquid media may likewise be introduced through inlet 5; however in this instance inlet 5 is desirably provided with a valve (not shown) which provides a larger inlet diameter (e.g. 3 to 8 mm) when liquid is to be introduced than the diameter (e.g. 0.2 to 2 mm, preferably 0.5 mm) used when gas is introduced. Liquid inlet 6 likewise conveniently has a diameter of 3 to 8 mm. Moreover about their periphery, first and second mixing chambers are provided with annular temperature control means, e.g. water cooling jackets, 31 and 32, temperature controlled by monitors 33,34 and control means 35,36. Further cooling means, e.g. coolant ducts, may be provided to cool the stators, the rotor drive shaft and the mechanical seal.

### Example 2

In a typical example of the use of the mixer apparatus of Example 1, drive shaft 11 is rotated at 8000 rpm or such a rate as to cause outer shear force zone 23 to have a relative rotor:stator speed of at least 32 m/s, e.g. 46 m/s.

Gold plated rotor:stator assemblies are novel and form a further aspect of the invention.

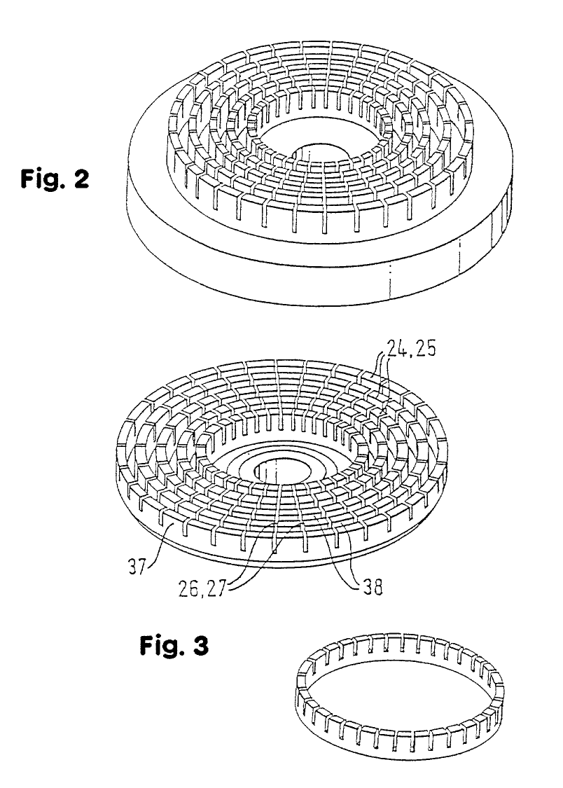

Referring to FIG. 2, it may be seen that the cylindrical extensions (flanges) 24,25 and axial slots 26,27 of the stators and rotors are axially extending and comprise a plurality of axially extending “teeth” 37 separated by a corresponding plurality of circumferentially evenly spaced, axially and radially extending apertures 38. In use, the materials being mixed pass through these apertures into successive shear force application zones defined by the circumferential sides of adjacent cylindrical extensions and axial slots.

Referring to FIG. 3, there is shown a representation of an individual flange 24,25.

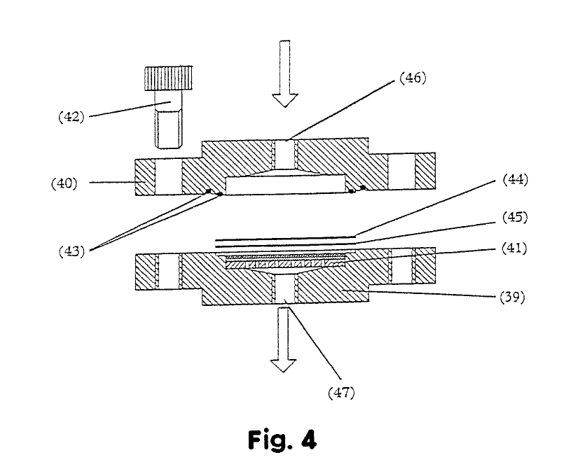

Referring to FIG. 4, there is shown a representation of a cross sectional drawing of an extruder suitable for use as a mixing apparatus according to the invention. Extrusion is performed using a pressurised reservoir (not shown) connected to a filter house with filters installed. The filter house assembly consists of the house bottom (39), the house lid (40), one or more support screens (41), bolts (42), o-ring seals (43) one or more filter membranes (44) and optionally one or more drain disks (45). Gold plating the parts which contact the fluid from the reservoir (other than the filter membranes (44) itself and particularly parts 39, 40 and 41 is beneficial for the efficiency of the extrusion operation. A liposome suspension may be introduced through inlet (46) at a pressure of up to approximately 300 bar. There are preferably three filter membranes (44) which may be formed from, for example, 2 μm polycarbonate sold under the Trade name Neopore by Corning. The drain disk (45) may be formed from a perforated porous sheet and which lies on top of the support screen(s) (41) which comprises a series of holes to allow the extruded liposome suspension to pass out of the extruder through outlet (47).

### Example 3

Rotor:stator assemblies as described, made of stainless steel 316 L or gold plated stainless steel 316 L were tested for yield (million/mL as determined using a Coulter Multisizer) of 3-5 μm microbubbles and for surface hydrophilicity (receding contact angle at 25 EC). The assemblies were untreated (U), steam sterilized for 1 hour (1S), unplated (316), gold plated (G), roughened (E), and/or steam sterilized for 15 production/sterilization cycles, each involving 1 hour of steam sterilization (15S). The yields and contact angles are set out below:

| Surface | Yield (million/mL) | Contact angle (E) |
|---|---|---|
| 316.U | 1200-2000 | 13 |
| 316.1S | 500 | 60 |
| 316.1SE | 500 | 70 |
| G.U | 1400 | 18 |
| G.1S | 1200 | 26 |
| G.15S | 1000 | 43 |

As can be seen, the gold plated assemblies did not suffer the rapid drop off in yield subsequent to sterilization associated with the unplated assemblies.

### Example 4

Liposome Extrusion

Using moderate pressure, e.g. 300 bar, a liquid crystalline multilamellar liposome suspension can be forced through filters with defined pore sizes. As the layers of the multilamellar liposome deform to pass through the pore, a breaking and resealing of the membranes occurs. If a liposome preparation is passed through filters many times, this process gives rise to a liposome population that reflects that of the filter pore.

Extrusion can be performed using an apparatus as described in the text above with reference to FIG. 4

Comparative Study:

In a comparative study, we have found that gold plating the parts 39, 40 and 41 is beneficial for the efficiency of the extrusion operation compared to an assembly with parts 39, 40 and 41 having surfaces of stainless steel. The filter membranes used were sets of three Nucleopore7 track-etched membrane filters supplied by Corning. The reservoir was pressurised until a steady flow came out of the filter house, and the pressure was gradually increased further if the flow significantly reduced.

| | Stainless Steel | Cold Plated |
|---|---|---|
| 1^{st} extrusion | 3 × 0.8 μm pore | 3 × 0.8 μm pore |
| stage | filters max. 10 barg | filters |
| | | max. 5 barg |
| 2^{nd} extrusion | 3 × 0.2 μm pore | 3 × 02 μm pore |
| stage | filters | filters |
| | max. 120 barg - | max. 25 barg |
| | decreasing flowrate | |
| 3^{rd} extrusion | 3 × 0.1 μm pore | 3 × 0.1 μm pore |
| stage | filters | filters |
| | max. 140 barg - full | max. 40 barg |
| | clogging | |
| Median size | 141 nm | 144 nm |

The table shows that the liposome suspension was much easier extruded in the gold plated extruder housing.

It is apparent that many modifications and variations of the invention as hereinabove set forth may be made without departing from the spirit and scope thereof. The specific embodiments described are given by way of example only, and the invention is limited only by the terms of the appended claims.

What is claimed is:

1. A process for the preparation of a mixture including a discontinuous phase in a continuous phase which comprises using a mixing apparatus having a non-porous mixing surface which is attractive to the continuous phase when the continuous phase is liquid or which is attractive to the discontinuous phase when the continuous phase is gaseous.

2. The process of claim 1 wherein said mixing surface is more hydrophilic after sterilisation than is stainless steel.

3. The process of claim 1 wherein said mixing surface is more hydrophobic after sterilisation than is stainless steel.

4. The process of claim 1 wherein the mixing surface is repellant to the discontinuous phase.

5. The process of claim 1 wherein said continuous phase is an aqueous liquid.

6. The process of claim 1 wherein said surface is made of or coated with gold or a ceramic material.

7. The process of claim 1 wherein said surface is plasma oven treated.

8. The process of claim 5 wherein water receding contact angle of said surface after steam sterilisation is less than 55 E.

9. The process of claim 1 wherein said mixture is a pharmaceutical composition.

10. The process of claims 1 wherein the continuous phase is an aqueous liquid and the discontinuous phase is at least partially gaseous.

11. The process of claim 1 wherein said mixing apparatus is selected from homogenizers, atomizers, sonicators, cap mixers, shakers, paddle mixers, blade mixers, static mixers, expansion nozzles, rotor stators, and extruders.

12. The process of claim 1 wherein said surface is steam sterilised before mixing.

13. The process of claim 1 for the preparation of a hydrophilic droplet-in-gas spray, wherein the mixing apparatus is a spray nozzle having a surface which is more hydrophilic than sterilized steel.

14. The process of claim 1 for the preparation of a hydrophobic droplet-in-gas spray, wherein the mixing apparatus is a spray nozzle having a surface which is more hydrophobic than sterilized steel.

15. A gold plated rotor:stator assembly.

16. An assembly comprising a sonicator horn or other actively vibrating surface placed in a sonicating chamber wherein at least part of the surfaces of said assembly are gold plated.

17. An extruder having a filter chamber comprising a filter membrane disposed on a support, characterised in that at least part of the surfaces within said chamber are gold plated.
